(19) United States
(12) Patent Application Publication
(10) Pub. No.: US 2008/0118931 A1
(43) Pub. Date: May 22, 2008

(54) Stabilized Ornithine Transcarbamylase and Immunoassay Method for Ornithine Transcarbamylase Using the Same

(75) Inventors: Hiroshi Murayama, Chiba (JP); Makoto Igarashi, Chiba (JP)

Correspondence Address: BELL, BOYD & LLOYD, LLP, P.O. Box 1135, CHICAGO, IL, 60690, US

(73) Assignee: YAMASA CORPORATION, Chiba (JP)

(21) Appl. No.: 11/813,053

(22) PCT Filed: Dec. 26, 2005
(86) PCT No.: PCT/JP05/23722

§ 371 Date: Jun. 28, 2007

(30) Foreign Application Priority Data
Jan. 7, 2005 (JP) 2006-002695

(51) Int. Cl. G01N 33/53; C12N 9/10

(52) U.S. Cl. 435/7.1; 435/193

(57) ABSTRACT

A method for stabilizing ornithine transcarbamylase (OTC) and a method of immunologically assaying OTC are provided. More specifically, the application provides a stabilized OTC solution having a pH of 5.5 to 7.0 as well as an immunological assay method of OTC including reacting an OTC antigen with an anti-OTC antibody at a pH of 7.5 to 10.5 or an immunological assay method of OTC including reacting an OTC antigen with an anti-OTC antibody at a pH of 6.5 to 10.5 in the presence of ProClin. According to the method of the present application, OTC level of a sample can be determined within a short period of time at high sensitivity. Thus, the method is useful for, for example, diagnosis of liver disease or follow-up after the onset of the disease.

### CROSS REFERENCE TO RELATED APPLICATIONS

The present application claims priority to Japanese Patent Application JP 2006-002695 filed with the Japan Patent Office on Jan. 7, 2005, the entire contents of which is being incorporated herein by reference.

### BACKGROUND

The present application relates to stabilized ornithine transcarbamylase; to a method for immunologically assaying ornithine transcarbamylase by use of the stabilized ornithine transcarbamylase; and to a kit employed for the assay method.

Ornithine transcarbamylase (OTC), which is also called “ornithine carbamoyltransferase (OCT)” or “citrulline phosphorylase,” is an enzyme useful for, for example, clinical diagnosis of liver disease or clinical follow-up after the onset of the disease.

As has been pointed out, immunological assay of OTC poses two serious problems: a problem in terms of stability of OTC serving as a standard substance; and a problem in terms of assay; i.e., requirement of a long-term antigen-antibody reaction for attaining sufficient assay sensitivity.

Specifically, isolated and purified native OTC, or recombinant OTC prepared through a DNA recombination technique, is a very unstable enzyme. Therefore, for stabilization, the enzyme is essentially subjected to a treatment, for example, causing the enzyme to coexist with glycerol (about 50%) (BioChem. J. 1997; 322: 625-631, J. Biol. Chem. 1978; 253: 3939-3944). However, difficulty is encountered in stabilizing OTC for a long period of time by merely glycerol. Meanwhile, as has been generally well known, an enzyme is stabilized by addition of a protein such as BSA or animal serum. However, OTC has failed to be stabilized for a long period of time by merely a protein.

Conventionally, immunological assay of OTC has been performed at a pH of 7.4 (i.e., almost neutral pH), which is comparable to that found in vivo (Enzyme Protein 1994-95; 48: 10-17, Enzyme Protein 1994-95; 48: 18-26). However, immunological assay at this pH requires an overnight primary reaction, a three-hour secondary reaction, and a 2.5-hour tertiary reaction; i.e., two days are required until test results are obtained.

In addition to the aforementioned problem, the conventional assay, which employs serum without dilution, poses a number of problems to be solved, including a concern regarding the effect of serum components on the assay.

Non-Patent Document 1: BioChem. J., 1997; 322: 625-631

Non-Patent Document 2: J. Biol. Chem., 1978; 253: 3939-3944

Non-Patent Document 3: Enzyme Protein, 1994-95; 48: 10-17

Non-Patent Document 4: Enzyme Protein, 1994-95; 48: 18-26

### SUMMARY

In general, an assay system of low sensitivity causes problems, including requirement of a long-term reaction, and large variation in data, resulting in failure to obtain reliable and accurate data in a consistent manner. In the case of an acute disease such as fulminant hepatitis, test results are required to be provided as soon as possible for rapid determination of therapeutic strategy, and a shorter period of time for assay is preferred. However, the conventional immunological assay method of OTC does not satisfy such requirements, and therefore demand has arisen for development of a method for accurately assaying OTC within a short period of time at high sensitivity in a consistent manner.

In the case where OTC is employed as a standard substance in a kit, when OTC itself exhibits poor stability, the kit fails to be stored for a long period of time, and thus the effective life of the kit is extremely shortened. Such a kit poses a problem in that it requires storage at a temperature of −40° C. or lower or similar measures, resulting in a poor level of user friendliness.

The present inventors have conducted extensive studies for solving problems involved in the conventional immunological assay method of OTC, and as a result have found that:

(1) when the pH of a buffer in which OTC is contained for preservation is adjusted so as to fall within an acidic pH range (specifically, 5.5 to 7.0), stability of OTC is increased considerably;

(2) when the OTC-containing buffer is caused to coexist with at least two species selected from among glycerol, a protein, a substrate for OTC, and an OTC reaction product (an analogue of the substrate or reaction product), stability of OTC is further increased;

(3) during immunological assay of OTC, when the pH of a reaction system is adjusted to a range of 7.5 to 10.5, reactivity of OTC with an antibody is increased; and

(4) when the reaction system is caused to coexist with ProClin (trade name), reactivity of OTC with the antibody is further increased within the aforementioned basic pH range, and immunological reactivity of OTC is increased within a neutral pH range; i.e., sufficient sensitivity can be obtained even within a pH range of 6.5 or higher.

Accordingly, the present application, which has been accomplished on the basis of these findings, provides the following.

[1] A stabilized OTC solution having a pH of 5.5 to 7.0.

[2] An OTC solution according to [2], which further contains glycerol, a protein, a substrate for OTC, an OTC reaction product, or an analogue of the substrate or reaction product.

[3] An OTC solution according to [2], which is in a liquid state, wherein the protein is a bovine-derived protein.

[4] An OTC solution according to [1], which is in a frozen state, wherein the protein is a milk-derived protein.

[5] A stabilized, lyophilized OTC product obtained through lyophilization of an OTC solution, wherein a protein and a sugar are incorporated as stabilizers during lyophilization.

[6] A lyophilized OTC product according to [5], wherein the OTC solution has a pH of 5.5 to 7.0 before lyophilization.

[7] A lyophilized OTC product according to [5], wherein the protein is a bovine-derived protein, and the sugar is a monosaccharide or a disaccharide.

[8] An immunological assay method of OTC, the method comprising reacting an OTC antigen with an anti-OTC antibody at a pH of 7.5 to 10.5.

[9] A method according to [8], wherein ProClin (trade name) is caused to coexist with an antigen-antibody reaction system.

[10] An immunological assay method of OTC, the method comprising reacting an OTC antigen with an anti-OTC antibody at a pH of 6.5 to 10.5 in the presence of ProClin.

[11] An OTC detection kit for assaying OTC through a method as recited in any of [8] to [10].

[12] A kit according to [11], which employs, as a standard substance, an OTC solution or a lyophilized OTC product as recited in any of [1] to [7].

As shown in the Examples described hereinbelow, the method of the present application has first enabled the OTC level of a sample to be determined at high sensitivity within a short period of time. Also, since the storage period of OTC is prolonged, a stable assay kit according to the present application can be provided. Thus, when the method of the present application is employed, the OTC level of a sample can be quantitatively determined accurately within a short period of time at high sensitivity in a consistent manner. That is, the method of the present application is useful for, for example, diagnosis of liver disease or follow-up after the onset of the disease.

Additional features and advantages of the present application are described in, and will be apparent from, the following Detailed Description and the figures.

### BRIEF DESCRIPTION OF THE FIGURES

FIG. 1 shows data of storage stability of recombinant OTC at 4° C. for 15 days.

FIG. 2 shows the effect of addition of ornithine or citrulline on storage stability at 4° C. for 10 days.

FIG. 3 shows an example of OTC standard curves employed in ELISA.

FIG. 4 shows the effect of pH (6.7) on reactivity of recombinant OTC or native OTC with an antibody in ELISA.

FIG. 5 shows the effect of pH (9.4) on reactivity of recombinant OTC or native OTC with an antibody in ELISA.

FIG. 6 shows the effect of pH on reactivity of native OTC with an antibody in the absence of ProClin.

FIG. 7 shows the effect of pH on reactivity of native OTC with an antibody in the presence of ProClin.

FIG. 8 shows the effect of ProClin concentration on reactivity of OTC with an antibody.

FIG. 9 shows the correlation between an enzyme activity assay method and ELISA.

### DETAILED DESCRIPTION

(1) Stabilization of OTC

No particular limitation is imposed on the OTC employed in the present application, so long as it is a mammalian hepatocyte-derived OTC.

Among such mammalian hepatocyte-derived OTCs, human hepatocyte-derived OTC employed may be in the form of native OTC; for example, human liver tissue extract available from BioChain, etc., or OTC isolated and purified from the extract through a customary method (J. Biol. Chem., 258 (18): 6464-6469 (1977), Arch. Biochem. Biophys., 309 (2): 293-299 (1994)).

Human hepatocyte-derived OTC has already been cloned, and the nucleotide sequence thereof has been known. Therefore, a large amount of OTC may be produced in a host (e.g., Escherichia coli) through a customary method by use of a cloned OTC gene, and OTC isolated and purified from cells of the microorganism may be employed as recombinant OTC (Science, 224: 1068-1074 (1984), J. BioChem., 103: 302-308 (1988), BioChem. J. 322: 625-631 (1997)).

A characteristic feature of the stabilized OTC solution of the present application resides in that it has a pH of 5.5 to 7.0. Such an OTC solution may be prepared by dissolving OTC in water having the aforementioned pH, preferably in a buffer having the aforementioned pH, such as a phosphate buffer, a Good's buffer (e.g., an MES-sodium hydroxide buffer), a citric acid-sodium phosphate buffer, a citric acid-sodium citrate buffer, an acetic acid-sodium acetate buffer, a β,β′-dimethylglutaric acid-sodium hydroxide buffer, a sodium cacodylate-hydrochloric acid buffer, a sodium maleate-sodium hydroxide buffer, or an imidazole-hydrochloric acid buffer, so that the OTC concentration is about 1 ng/mL to about 100 μg/mL.

The OTC solution may contain a stabilizer such as glycerol, a protein, a sugar, a substrate for OTC, an OTC reaction product, or an analogue of the substrate or reaction product, for the purpose of further improving stability of the OTC solution.

Although glycerol which is commercially available as a reagent may be employed as a stabilizer, highly purified glycerol is preferably employed.

The protein may be a protein which is generally used as an enzyme stabilizer. Examples of preferably employed proteins include bovine blood-derived proteins such as BSA, FCS, and bovine serum; and milk-derived proteins such as skim milk, casein, and Block Ace (trade name).

Examples of the sugar which may be employed include monosaccharides such as glucose, mannose, galactose, and fructose; and disaccharides such as sucrose, lactose, maltose, and trehalose.

Examples of the substrate for OTC or the OTC reaction product include ornithine and citrulline. Examples of the analogue of the substrate or reaction product which may be employed include norvaline (a substrate analogue), N^{δ}-(phosphonacetyl)-L-ornithine, and arginine phosphate.

These stabilizers may be employed independently, but are preferably employed in combination of two or more species. Employment of two or more stabilizers in combination enables OTC to be stabilized for a longer period of time. No particular limitation is imposed on, for example, the combination of stabilizers employed and the concentrations of the stabilizers, and these factors may be appropriately determined through a small-scale test. An antiseptic such as sodium azide may be employed in combination with the stabilizers.

The OTC of the present application may be in a liquid state as described above, or may be in a frozen or lyophilized state.

Frozen OTC may be prepared by adding, to OTC, a stabilizer; in particular, a protein (preferably, a milk-derived protein such as skim milk, casein, or Block Ace) before freezing so as to prevent denaturation through freezing, followed by freezing treatment of the resultant mixture.

Lyophilized OTC may be prepared by preparing an OTC solution having a pH of 5.5 to 7.0, and then adding a stabilizer (preferably, two stabilizers (i.e., a protein and a sugar)) to the OTC solution, followed by lyophilization treatment of the resultant mixture through a customary method.

(2) Immunological Assay Method and Kit

A characteristic feature of the immunological assay method of the present application resides in that, in the absence of ProClin, the pH of a reaction system is adjusted to a range of 7.5 to 10.5 (preferably 8.2 to 10.2), whereas in the presence of ProClin, the pH of a reaction system is adjusted to a range of 6.5 to 10.5 (preferably 7.0 to 10.2). As described in the Examples hereinbelow, when reaction is performed under such pH conditions, reactivity of OTC with an antibody is increased, and thus the OTC level of a sample can be accurately determined within a short period of time.

ProClin, which is added to a reaction system, is generally employed as an antiseptic in clinical diagnosis. Four types of ProClin are commercially available; i.e., ProClin 150, ProClin 200, ProClin 300, and ProClin 950. Any of these types contains, as an active ingredient, isothiazolone (5-chloro-2-methyl-4-isothiazolin-3-one and/or 2-methyl-4-isothiazolin-3-one). When ProClin is employed as an antiseptic, the recommended concentration is 6 to 15 ppm (ProClin 150, ProClin 200, or ProClin 300), or 48 to 95 ppm (ProClin 950). In the present application, as described in the Examples hereinbelow, ProClin may be employed at a concentration within the aforementioned recommended concentration range or at a concentration above the range.

The immunological assay method or kit of the present application may be carried out or prepared through a known method, except that an OTC antigen is reacted with an antibody under the aforementioned pH conditions.

Examples of test samples employed include, but are not particularly limited to, a serum sample or plasma sample from a patient with suspected liver disease.

No particular limitation is imposed on the antibody employed in the present application, so long as it is an antibody reactive with OTC (i.e., OTC-reactive antibody). The antibody may be a monoclonal antibody or a polyclonal antibody. The present application may employ such an antibody per se, or an active fragment thereof (e.g., F(ab′)₂ or Fab′).

As used herein, the term “OTC-reactive antibody” refers to an antibody which can be bound to both native OTC and recombinant OTC, and is not limited to an antibody having specific properties.

Such an OTC-reactive antibody may be prepared through a method known in the literature. For example, such an antibody may be selected from polyclonal antibodies or monoclonal antibodies which are reactive with recombinant OTC serving as an antigen, through screening in accordance with a conventional procedure.

As described above, the procedure, etc. of the immunological assay method employing such an antibody are the same as those of a conventional immunological assay method, except for pH conditions.

Examples of the antibody for trapping OTC contained in a sample include an antibody bound onto a support (i.e., an immobilized antibody).

Examples of the support employed for preparing such an immobilized antibody include generally employed supports, including synthetic organic polymer compounds such as polyvinyl chloride, polystyrene, styrene-divinylbenzene copolymers, styrene-maleic anhydride copolymers, nylon, polyvinyl alcohol, polyacrylamide, polyacrylonitrile, polypropylene, and polymethylene methacrylate; polysaccharides such as dextran derivatives (e.g., Sephadex), agarose gel (e.g., Sepharose or Biogel), and cellulose (e.g., paper disk or filter paper); and inorganic polymer compounds such as glass, silica gel, and silicone. Such a support may have an introduced functional group (e.g., an amino group, a carboxyl group, a carbonyl group, a hydroxyl group, or a sulfhydryl group).

Such a support may assume any form, such as a plate form (e.g., microtiter plate or disk), a particulate form (e.g., beads), a tubular form (e.g., test tube), a fibrous form, a membrane form, a microparticulate form (e.g., latex particles), a capsule form, or an endoplasmic reticulum form. A support of suitable form may be appropriately selected in consideration of the assay method employed.

Binding of an antibody to a support may be carried out through a known technique such as physical adsorption, ionic binding, covalent binding, or entrapment [see, for example, “Koteika Koso” (“Immobilized Enzyme”) (edited by Ichiro Senhata, published by Kodansha Ltd. on Mar. 20, 1975)]. Particularly, physical adsorption is preferred, from the viewpoint of convenience. An antibody may be bound directly to a support, or a substance may be provided between an antibody and a support.

In order to suppress non-specific binding, the thus-prepared immobilized reagent may be subjected to blocking treatment by use of a generally employed blocking agent such as gelatin, BSA, or skim milk.

Examples of the antibody for detecting trapped OTC include an antibody labeled with a labeling agent.

Examples of labeling agents employed include radioisotopes such as ³²P, ³H, ¹⁴C, and ¹²⁵I; enzymes such as β-galactosidase, peroxidase, alkaline phosphatase, glucose-6-phosphate dehydrogenase, catalase, glucose oxidase, lactate oxidase, alcohol oxidase, and monoamine oxidase; coenzymes and prosthetic groups such as FAD, FMN, ATP, biotin, and heme; fluorescein derivatives such as fluorescein isothiocyanate and fluorescein thiofurbamyl; rhodamine derivatives such as tetramethylrhodamine B isothiocyanate; fluorescent dyes such as umbelliferone and 1-anilino-8-naphthalenesulfonate; and luminol derivarives such as luminol, isoluminol, and N-(6-aminohexyl)-N-ethylisoluminol.

Binding of an antibody to a labeling agent may be carried out through a method appropriately selected from among known methods described in the literature [for example, “Zoku Seikagaku Jikken Koza 5, Men-eki Seikagaku Kenkyu-ho” (“Sequel to Biochemical Experiments 5, Immunobiochemical Studies”), published by Tokyo Kagaku Dojin Co., Ltd. (1986), pp. 102-112].

Assay employing such an immobilized antibody and labeled antibody may employ a typical immunoassay procedure as it is. Specifically, an immobilized antibody is reacted with a test sample, followed by optional BF separation, and the resultant product is reacted with a labeled antibody (two-step method), or an immobilized antibody, a test sample, and a labeled antibody are reacted simultaneously (one-step method); and subsequently, OTC contained in the sample is detected or quantitatively determined through a method which is known per se.

Details of immunoassay are described in, for example, the following references.

(1) “Zoku Rajio Immunoassei” (“Sequel to Radioimmunoassay”) edited by Hiroshi Irie (published by Kodansha Ltd. on May 1, 1979)

(2) “Koso Men-eki Sokutel-ho” (“Enzyme Immunoassay”) (2nd Edition) edited by Eiji Ishikawa, et al. (published by Igaku-Shoin Ltd. on Dec. 15, 1982)

(3) Rinsho Byori Extra Edition No. 53 “Rinsho Kensa no tameno Immunoassei—Gijutsu to Oyo—(“Immunoassay for Clinical Tests—Techniques and Applications-”) (published by The Clinical Pathology Press, 1983)

(4) “Biotechnology Encyclopedia” (published by CMC Publishing Co., Ltd. on Oct. 9, 1986)

(5) “Methods in ENZYMOLOGY Vol. 70” (Immunochemical techniques (Part A))

(6) “Methods in ENZYMOLOGY Vol. 73” (Immunochemical techniques (Part B))

(7) “Methods in ENZYMOLOGY Vol. 74” (Immunochemical techniques (Part C))

(8) “Methods in ENZYMOLOGY Vol. 84” (Immunochemical techniques (Part D: Selected Immunoassay)) (9) “Methods in ENZYMOLOGY Vol. 92” (Immunochemical techniques (Part E: Monoclonal Antibodies and General Immunoassay Methods))

[(5) to (9), published by Academic Press]

A characteristic feature of the kit employed for the aforementioned immunological assay resides in that the kit contains, as its constituent reagent (which serves as a standard substance), OTC stabilized in a buffer having a pH of 5.5 to 7.0 (or a lyophilized product of the OTC), wherein an antigen-antibody reaction is carried out at a pH of 7.5 to 10.5 (or 6.5 to 10.5 in the presence of ProClin). The kit may appropriately contain an additional constituent reagent which is required for an assay employed. Specifically, for example, a kit for carrying out ELISA may contain the following constituent reagents.

(1) OTC (standard substance) having a predetermined concentration

(2) Anti-OTC antibody-immobilized plate

(3) Sample diluent

(4) Peroxidase-labeled anti-OTC antibody

(5) Labeled antibody diluent

(6) Washing liquid

(7) Color developing liquid

(8) Color development stopping liquid

In the aforementioned kit, the concentration-specified OTC (standard substance) may be contained in a buffer having a pH of 5.5 to 7.0, or may first be dissolved in the buffer, followed by lyophilization. Meanwhile, for example, a buffer having high buffering capacity may be employed as the sample diluent or the labeled antibody diluent so that the final pH of the assay system is adjusted to 7.5 to 10.5.

### EXAMPLES

The present application will next be described in detail by way of examples, which should not be construed as limiting the application thereto. All procedures, including preparation of DNA, cleavage with restriction enzymes, ligation of DNA by T4 DNA ligase, and transformation of Escherichia coli, were performed according to “Molecular Cloning” (edited by Maniatis, et al., Cold Spring Harbor Laboratory, Cold Spring Harbor, N.Y. (1982)). Restriction enzymes, LATaq DNA polymerase, and T4 DNA ligase were obtained from Takara Bio Inc.

### Example 1

### Stabilization of OTC

(1) Preparation of OTC cDNA

There was employed, as human OTC cDNA, OTC cDNA which had been artificially synthesized from mRNA prepared from the liver through a known recombinant DNA technique (Science, 224: 1068-1074 (1984), J. BioChem., 103: 302-308 (1988), BioChem. J. 322: 625-631 (1997)). Specifically, human liver-derived cDNA library (purchased from Clontech and Takara Bio Inc.) was amplified according to the manual attached thereto by infecting Escherichia coli with recombinant phage, followed by deproteinization through treatment with protease K (0.2 mg/mL, 37° C., 60 minutes) and with phenol, and then recombinant phage DNA was precipitated by ethanol. The thus-precipitated DNA was dissolved in sterile water, to thereby prepare a recombinant phage solution. In order to amplify and isolate OTC cDNA from the cDNA library through PCR, the below-described two primer DNAs were synthesized.

As has been reported, immature OTC protein has, on the N-terminal side, a mitochondrial transport signal formed of 32 amino acid residues. Therefore, in order to remove this amino acid sequence, the below-described sense primer (A) was synthesized so that a BamHI site was provided at a position before the N-terminal (33rd) amino acid residue of mature OTC protein (i.e., asparagine), so as to attain cloning into expression vector pQE31. Similarly, the antisense primer (B) was synthesized so that an XbaI site was provided at the C-terminal sequence of OTC protein and on the 5′-side of the TGA stop codon, so as to attain cloning into pQE31. By using, as a template, the phage DNA solution prepared from the human liver-derived cDNA library, human liver-derived OTC cDNA (Submitted to NCBI, Accession No. K02100) was amplified through PCR by use of the aforementioned two primers.

Primer (A):
5′-CAA CCG GAT CCA AAT AAA GTG CAG CTG AAG-3′
Primer (B):
5′-AAC TCT AGA TCA AAA TTT AGG CTT CTG GAG-3′

PCR amplification of the OTC cDNA was performed by means of a DNA Thermal Cycler personal (product of Takara Bio Inc.) through 30 cycles of treatment, each cycle consisting of the steps of thermal denaturation (94° C., one minute), annealing (59° C., one minute), and elongation (72° C., two minutes), of a mixture (final volume: 50 μL) containing LATag DNA Polymerase (product of Takara Bio Inc.), 10×LAPCR buffer (5 μL), 25 mM MgCl₂ (5 μL), dNTP (8 μL), primer DNAs (A) and (B) (10 μmol each), and a DNA sample (about 0.5 μg).

After amplification of the gene, the resultant DNA was separated through agarose gel electrophoresis according to the method of the literature (“Molecular Cloning”) to thereby purify a DNA fragment of 1 kb. The DNA fragment was cleaved with restriction enzymes BamHI and XbaI, and the thus-formed fragment was cloned into plasmid pUC118 (purchased from Takara Bio Inc.), which had been digested with restriction enzymes BamHI and XbaI. The nucleotide sequence of the thus-cloned DNA fragment was determined through a routine method. The determined nucleotide sequence completely corresponded to the nucleotide sequence of OTC cDNA reported by Akira Hata, et al. (J. BioChem., 103: 302-308 (1988)), and thus the cloned DNA fragment was identified as an OTC gene. Subsequently, the DNA fragment was cleaved with Bam-HI and SalI, and ligated with pQE31 (purchased from Qiagen)—which had been cleaved with the same restriction enzymes BamHI and SalI—by use of T4 DNA ligase, to thereby form pQE31:OTC plasmid. Escherichia coli K12 strain JM109 (purchased from Takara Bio Inc.) was transformed by use of the resultant ligation reaction mixture, and the plasmid pQE31:OTC was isolated from the thus-yielded ampicillin-resistant transformant cells.

H. Morizono et al. have reported that in the case of expression of human OTC protein in Escherichia coli, when GroEL and GroES genes are coexpressed, formation of an inclusion body is suppressed in Escherichia coli cells, and OTC protein with high activity is collected (Biochem. J. (1997) 322). Therefore, by using, as a template, Escherichia coli chromosomal DNA prepared through a customary method, the below-described two primer DNAs were synthesized through a customary method, and an Escherichia coli GroESL gene (Submitted to NCBI, Accession No. AAC77102-3) was amplified through PCR.

The below-described primer (C) (sense primer) was synthesized by use of a sequence upstream of an upstream promoter-containing region of the GroES gene. Meanwhile, in the case of the primer (D) (antisense primer), a sequence complementary to a region downstream of the stop codon of the GroEL gene was employed. Therefore, the fragment amplified from Escherichia coli chromosomal DNA by use of the primers (C) and (D) contains 2317 nucleotides including coding regions of both the GroES and GroEL genes and promoters thereof.

Primer (C):
5′-CAT GGG TTG ATG TCC GAT TG-3′
Primer (D):
5′-AAC CCC CAG ACA TTT CTG CC-3′

After amplification of the gene, the resultant DNA was separated through agarose gel electrophoresis according to the method of the literature (“Molecular Cloning”) to thereby purify a DNA fragment of about 2.3 kb. The DNA fragment was cloned into pUC118 plasmid (purchased from Takara Bio Inc.), and then the cloned fragment was cleaved with restriction enzymes HindIII and SmaI, followed by ligation with plasmid pACYC184 (purchased from New England Biolabs)—which had been digested with restriction enzymes BamHI and EcoRV—by use of T4 DNA ligase, to thereby form a pACYC:GroE plasmid. Escherichia coli JM109 was transformed by use of the resultant ligation reaction mixture, and the plasmid pACYC:GroE was isolated and purified from the cells of the thus-produced chloramphenicol-resistant transformant.

The expression vector pQE31 contains six histidine codons following the start codon, and thus six histidine residues (histidine tag) are added to the N-terminus of an expressed recombinant protein. Such a histidine tag, which is known to be adsorbed onto divalent ions of nickel or zinc, is employed in recombinant protein purification methods. For extraction and purification of OTC protein having a histidine tag at the N-terminus, Escherichia coli JM109 was transformed with the plasmids pQE31:OTC and pACYC:GroE, to thereby yield a transformant strain JM109/pQE31:OTC,pACYC:GroE.

The transformant cells were inoculated into an LB medium (20 mL) containing ampicillin (100 mg/L) and chloramphenicol (10 mg/L), followed by preliminary culturing at 37° C. overnight. Thereafter, the entire culture was inoculated into an LB medium (500 mL) containing ampicillin (100 mg/L) and chloramphenicol (10 mg/L), followed by further shaking culture at 37° C. Culturing was continued, and at the time when OD600 reached 1.0, IPTG was added so that the final concentration thereof was 0.01 mM, followed by shaking culture at 25° C. overnight. After completion of shaking culture, cells were isolated through centrifugation, and then suspended in an extraction buffer (50 mM Tris-HCl (pH 8.0), 50 mM NaCl, 1 mM EDTA, 1 mM PMSF, 2 μg/mL leupeptin, 2 μg/mL pepstatin, 4 μg/mL aprotinin) (50 mL), followed by ultrasonic disruption under ice cooling. The cell suspension formed through ultrasonication was centrifuged, and the residue was separated. Thereafter, glycerol was added to the thus-collected supernatant so that the final concentration thereof was 20%.

The protein concentration was determined, and a prepared sample (about 10 mg) was applied to a Histrap HP column (5 mL) (Amersham Bioscience). Subsequently, the column was washed with 20 mL of PBS, 20% glycerol, and with 10 mL of 80 mM imidazole, PBS, 20% glycerol, to thereby elute contaminant proteins. Thereafter, OTC protein was eluted and purified by use of 10 mL of 300 mM imidazole, PBS, 20% glycerol.

The thus-purified OTC protein was detected as an almost single band (molecular weight: about 36,000) through SDS-PAGE. The purified OTC protein was found to have an OTC enzyme activity, and was found to exhibit reactivity with the below-described anti-OTC monoclonal antibody through Western blotting. The purified protein concentration was quantitatively determined through the Lowry method employing bovine serum albumin as a standard.

In order to confirm that the histidine tag does not affect immune response, recombinant OTC having no histidine tag was prepared in a manner similar to that described above. Specifically, a DNA fragment amplified by use of the below-described two primers was ligated, in the form of an NcoI-BamHI fragment, with pT7B vector, and Escherichia coli JM109 was transformed by the ligated product for the expression of OTC. The thus-yielded OTC per se was employed without purification of the extract of the cells.

Primer (E):
5′-CAA CCC ATG GGA AAT AAA GTG CAG CTG AAG-3′
Primer (F):
5′-AAC TCT AGA TCA AAA TTT AGG CTT CTG GAG-3′

The thus-yielded recombinant OTC and liver-derived native OTC were employed for the below-described studies on factors affecting stabilization of OTC. Human liver tissue extract (BioChain) was employed as human liver-derived native OTC.

(2) Factors Affecting Stabilization of OTC

(2-1) Effect of pH

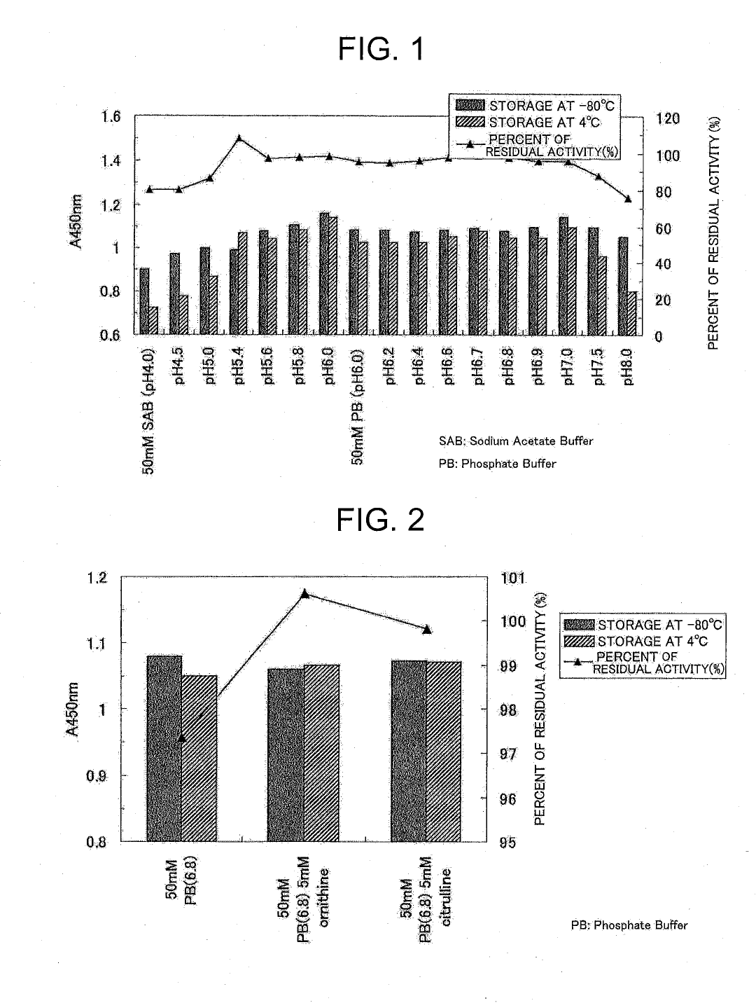

Stability of OTC in an aqueous solution was investigated. The results are shown in FIG. 1. As is clear from FIG. 1, within a pH range of 5.0 or lower and that of 7.5 or higher, OTC lost its immunoactivity by 10% or more after storage at 4° C. for 15 days, whereas within a pH range of 5.5 to 7.0, OTC retained its immunoactivity for 15 days.

(2-2) Effect of Ornithine or Citrulline

Ornithine (i.e., a substrate for OTC) or citrulline (i.e., an OTC reaction product) was added to an aqueous OTC solution (pH 6.8). As is clear from FIG. 2, OTC was stabilized through addition of such a substance, as compared with the case where no such substance was added. Similar to the case of such a substance, norvaline (i.e., a substrate analogue) or a similar substance was found to stabilize OTC.

(2-3) Effect of Antiseptic

Addition of sodium azide to an aqueous OTC solution did not affect reactivity of OTC.

(2-4) Effect of Glycerol, Protein, or Ornithine (or Citrulline)

Long-term storage stability of an OTC solution was investigated. A storage stability test (4° C., 10 days) showed that OTC was stabilized through addition of any one of glycerol, a protein (e.g., BSA), ornithine, and citrulline, as compared with the case where no such substance was added. However, a long-term stability test (six months) showed that such a substance did not necessarily exhibit satisfactory OTC-stabilizing effect.

Therefore, two or more species of these substances were added in combination, and the effect of the combination on stabilization of OTC was studied in a liquid state, a frozen state, or a lyophilized state. The results are shown in Table 1 shown below. As is clear from Table 1, in the case of addition of BSA alone (No. 1), percent activity retention of OTC was 78% under storage at 10° C. or −20° C. In the case of addition of glycerol alone (No. 2), OTC retained its activity sufficiently at a high concentration, but exhibited considerably reduced activity at a low concentration (in particular, 100 ng/mL or less).

In contrast, in the case of addition of 0.1% (w/v) BSA and 10% (w/v) glycerol in combination (No. 3), percent activity retention of OTC was improved to 86% or 92%, respectively. In the case of addition of BSA, glycerol, and ornithine in combination (No. 4), percent activity retention of OTC was improved to 88% or 96%, respectively. Data of No. 3 and No. 5 revealed that BSA is suitable for OTC stabilization in a liquid state, whereas skim milk is suitable for OTC stabilization in a frozen state. Although not shown in Table 1, similar results were obtained in the case of addition of bovine serum (i.e., a protein), citrulline (in place of ornithine), or norvaline (i.e., a substrate analogue). That is, any of these substances was found to serve as a stabilizer.

Data of No. 6 and No. 7 of Table 1 indicated that stability of OTC in a lyophilized state is improved through use of BSA and sucrose in combination.

(Table 1) Recombinant OTC Long-Term Stability Test (Storage for Six Months)

TABLE 1

| | | | Storage | Percent activity |
|---|---|---|---|---|
| | | | temperature | retention |
| No. | Diluent composition | State | (° C.) | (%) |
| 1 | 50 mM PB (pH 6.7), 50 mM NaCl, | Liquid | 10° C. | 78 |
| | 0.05% NaN₃, 0.1% BSA | Frozen | −20° C. | 78 |
| 2 | 50 mM PB (pH 6.7), 50 mM NaCl, | Liquid | 10° C. | 0 (low concentration) |
| | 0.05% NaN₃, 10% glycerol | Frozen | −20° C. | 0 (low concentration) |
| 3 | 50 mM PB (pH 6.7), 50 mM NaCl, | Liquid | 10° C. | 86 |
| | 0.05% NaN₃, 0.1% BSA, 10% | Frozen | −20° C. | 92 |
| | glycerol | | | |
| 4 | 10 mM PB (pH 6.7), 50 mM NaCl, | Liquid | 10° C. | 88 |
| | 0.05% NaN₃, 0.1% BSA, 5% | Frozen | −20° C. | 96 |
| | glycerol, 10 mM ornithine | | | |
| 5 | 10 mM PB (pH 6.7), 50 mM NaCl, | Liquid | 10° C. | 50 |
| | 0.05% NaN₃, 0.1% skim milk, | Frozen | −20° C. | 97 |
| | 10% glycerol | | | |
| 6 | 10 mM PB (pH 6.7), 50 mM NaCl, | Lyophilized | 10° C. | 77 (three months) |
| | 0.05% NaN₃, 0.1% BSA | | | |
| 7 | 10 mM PB (pH 6.7), 50 mM NaCl, | Lyophilized | 10° C. | 96 |
| | 0.05% NaN₃, 0.1% BSA, 5% | | | |
| | sucrose | | | |

### Example 2

### Immunological Assay of OTC

(1) Preparation of Anti-OTC Monoclonal Antibody

Recombinant OTC (50 μg) and complete Freund's adjuvant were intraperitoneally administered to BALB/c mice, ranging in age from six to eight weeks old, four times every two to three weeks. Two weeks after the fourth immunization, blood was drawn from the mice, and antibody titer to OTC was determined through ELISA as follows.

Specifically, recombinant human OTC was diluted with PBS to 1 μg/mL, and then dispensed into a flexible assay plate (product of Falcon) (50 μL/well), followed by allowing the plate to stand still at 4° C. overnight. After the plate washed with PBS three times, a 0.5% skim milk solution was dispensed into the plate (200 μL/well), and then the plate was allowed to stand still at room temperature for one hour. The skim milk solution was removed, and mouse serum which had been diluted stepwise with 1% BSA-containing PBS was dispensed into the plate (50 μL/well), followed by allowing the plate to stand still at room temperature for one hour. After the plate was washed with PBS three times, a solution prepared by diluting HRP-labeled goat anti-mouse IgG antibody (product of Zymed) with 1% BSA-containing PBS to 1/1,000 was dispensed into the plate (50 μL/well), and the plate was allowed to stand still at room temperature for one hour. After the plate was washed with PBS three times, a substrate solution (0.3 mM 3,3′,5,5′-tetramethylbenzidine dihydrochloride, 0.2 M citrate buffer containing 0.005% aqueous hydrogen peroxide, pH 3.8) was dispensed into the plate (100 μL/well), and the plate was allowed to stand still at room temperature for five minutes for color development. Reaction was terminated by adding 1 N sulfuric acid to the plate (100 μL/well), and absorbance at 450 nm was measured by means of a microplate photometer.

Recombinant OTC (10 μg) dissolved in saline was intravenously administered to each of the mice for final immunization. Three days after the final immunization, the spleen was extirpated from the mouse, and spleen cells were fused with mouse myeloma cells Sp2/0-Ag14 (Sp2) (ATCC CRL-1581) through the method of Koehler and Milstein. Specifically, spleen cells and myeloma cells were mixed at a ratio of 10:1, and the cell mixture was subjected to centrifugation. To the thus-obtained pellets was gradually added an RPMI1640 solution (1 mL) containing 50% polyethylene glycol for cell fusion. RPMI1640 medium was added to the resultant product so as to attain a total volume of 10 mL, and the mixture was subjected to centrifugation. The thus-obtained pellets were suspended in RPMI1640 medium containing 10% fetal calf serum (FCS) so that the concentration of the Sp2 cells was 3×10⁴ cells/100 μL, and the resultant suspension was dispensed into ten 96-well microtiter plates (100 μL/well).

One day later, HAT medium was added to the plate (100 μL/well), and the half of the medium was replaced by fresh HAT medium every three or four days. On the seventh day after the fusion, the culture supernatant was sampled, and screening was performed by use of the culture supernatant in place of mouse serum employed in the aforementioned ELISA. Wells positive for an antibody to OTC (i.e., an anti-OTC antibody) were retrieved, and cloning was performed through limiting dilution. Through two cycles of cell fusion, hybridomas producing an anti-OTC antibody were established (total: 22 clones). The hybridomas were dispensed into glass vials, and then cryopreserved in liquid nitrogen.

Each of the thus-established hybridomas was cultured, and then intraperitoneally administered to mice which had received pristane in advance (3×10⁶ cells/mouse). Eight to fourteen days later, ascites was collected from each of the mice. The thus-collected ascites and 1.5 M glycine-HCl buffer containing 3 M sodium chloride (pH 8.9) were mixed at a ratio of 1:1, and the resultant mixture was subjected to filtration with a 0.22-atm membrane filter, followed by application to a Protein A Sepharose CL-4B column (product of Pharmacia) equilibrated with the aforementioned buffer. After the column was washed with a sufficient amount of the buffer, an antibody was eluted with 0.1 M citrate buffer (pH 6.0). The resultant eluate was dialyzed against PBS, and the purity of the antibody was determined through SDS-polyacrylamide gel electrophoresis. The antibody was provided as a purified monoclonal antibody.

(2) Enzyme Labeling of Monoclonal Antibody

The thus-obtained purified monoclonal antibody (20 mg) was dialyzed against 0.1 M sodium citrate buffer (pH 3.9) (2 L) at 4° C. overnight. Pepsin was dissolved in 0.1 M sodium citrate buffer (pH 3.9) so as to attain a concentration of 1 mg/mL. The pepsin solution (100 μL) was added to a 10 mg/mL antibody solution (2 mL), and the resultant mixture was allowed to stand still at 37° C. for 16 hours. After being neutralized with 3 M Tris buffer (pH 8.8) (200 μL), the mixture was applied to a Sephacryl S-200HR column (1×45 cm, 35 mL, PBS), and fractions (0.7 mL each) were obtained at 20 mL/h while absorbance at 280 nm (A280) was monitored. The fraction corresponding to the initial peak was collected as an F(ab′)₂ fraction.

The thus-collected F(ab′)₂ fraction was dialyzed against 0.01 M sodium carbonate buffer (pH 9.5) (2 L) at 4° C. overnight. Horseradish peroxidase (HRPO: product of Toyobo Co., Ltd.) was dissolved in distilled water so as to attain a concentration of 4 mg/mL. 0.1 M Sodium periodate solution (100 μL) was added to the HRP solution (0.5 mL), and the resultant mixture was allowed to stand still at room temperature for exactly 20 minutes. The mixture was dialyzed against 1 mM sodium acetate buffer (pH 4.0) (2 L) at 4° C. overnight, and subsequently 0.2 M sodium carbonate buffer (pH 9.5) (about 25 μL) was added to the mixture, to thereby adjust the pH to a range of 9.0 to 9.5. The antibody solution (0.5 mL) was added to the HRP solution (0.5 mL) in a serum tube, and the resultant mixture was slowly stirred at room temperature for two hours. Sodium borohydride (4 mg/mL) (50 μL) was added to the mixture, and the mixture was allowed to stand still at 4° C. for two hours, followed by dialysis against PBS (2 L) overnight, to thereby yield an enzyme-labeled antibody.

(3) OTC Assay Through ELISA

(3-1) Standard Curve

An anti-OTC monoclonal antibody (3B11 or 6H11) was diluted with PBS so as to attain a concentration of 10 μg/mL, and a microtiter plate was coated with the PBS-diluted antibody (100 μL/well). The plate was allowed to stand still at 25° C. overnight, and then washed three times with PBS containing 0.05% Tween 20. Thereafter, the plate was subjected to blocking treatment; i.e., a blocking solution (0.5% skim milk, 5% sucrose, 0.1% ProClin 300) was dispensed into the plate (300 μL/well), and the plate was allowed to stand still at 25° C. for one hour.

After the blocking solution had been removed, an HRP-labeled anti-OTC monoclonal antibody (F(ab′)₂ of 5B11 or 4G6) was diluted with 0.25 M glycine-NaOH buffer (pH 9.4) containing 0.1% Tween 20, 0.1% BSA, 150 mM NaCl, and 0.1% ProClin 950 so that the antibody concentration was 1 μg/mL, and the buffer-diluted antibody was dispensed into the plate (50 μL/well). Subsequently, a sample was diluted with a sample diluent (0.1% Tween 20, 0.1% BSA, 150 mM NaCl, 10 mM PBS (pH 8.0), 0.1% ProClin 950) to 1/10, and the thus-diluted sample was dispensed into the plate (50 μL/well).

After one-minute stirring, the plate was allowed to stand still at room temperature for two hours, and then the plate was washed three times with a washing liquid (0.1% BSA, 150 mM NaCl, 0.1% ProClin 950, 10 mM glycine-NaOH buffer (pH 9.4)). Thereafter, a color developing liquid (TMBZ) was dispensed into the plate (100 μL/well), and the plate was allowed to stand still at room temperature for 15 minutes for color development. Color development was stopped by dispensing 1 N sulfuric acid into the plate (100 μL/well), and then absorbance at 450 nm was measured. A standard was added to the plate in parallel with the sample, and the OTC level of the sample was calculated on the basis of a standard curve.

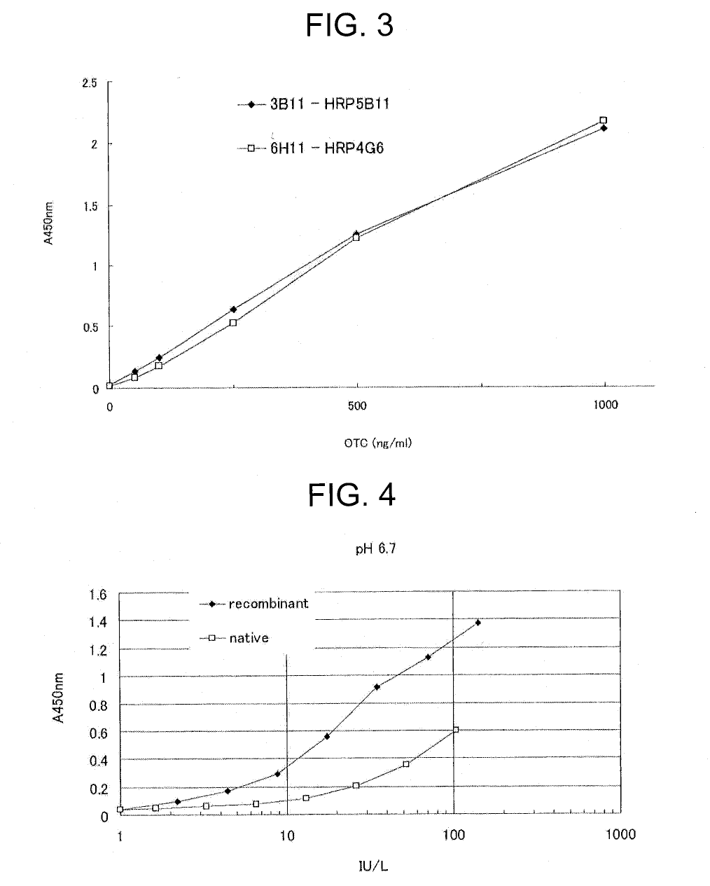

Recombinant OTC standard liquids were assayed through the aforementioned procedure. The results are shown in FIG. 3. FIG. 3 shows data of two assay systems; i.e., an assay system employing 3B11 as an immobilized antibody and 5B11(F(ab′)₂) as a labeled antibody; and an assay system employing 6H11 as an immobilized antibody and 4G6(F(ab′)₂) as a labeled antibody. As is clear from FIG. 3, similar standard curves are obtained from these assay systems, and thus any of these antibodies, which are reactive with OTC, can be employed for OTC assay.

(3-2) Reactivity with Native OTC

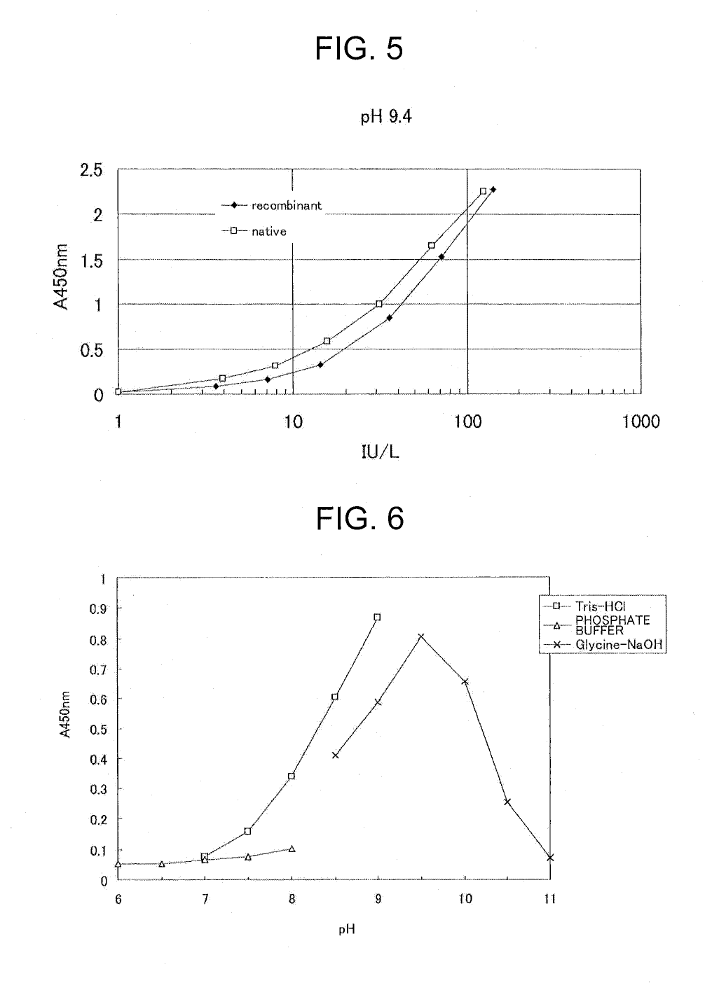

Difference between reactivity of an antibody with recombinant OTC and that of the antibody with native OTC was studied by employing a human liver tissue extract as native OTC, and by comparing ELISA reactivity per unit enzyme activity of native OTC with that of recombinant OTC. The results are shown in FIGS. 4 and 5. As shown in data of FIG. 4, when the pH of the assay system is adjusted to 6.7, reactivity of the antibody with native OTC is reduced to about 1/10 of that of the antibody with recombinant OTC. In contrast, as shown in data of FIG. 5, when the pH of the assay system is adjusted to 9.4, reactivity of the antibody with native OTC is almost equal to that of the antibody with recombinant OTC.

(4) Studies on Enhancement in Sensitivity of Assay System

(4-1) Effect of pH on Assay System

The effect of pH on reactivity of an antibody with native OTC was studied by varying the pH of an assay system (immobilized antibody: 3B11, labeled antibody: 5B11); specifically, the pH of a buffer employed for diluting the labeled antibody. The results are shown in FIG. 6. As is clear from FIG. 6, good reactivity is obtained within a pH range of 7.5 to 10.5. In the case of recombinant OTC, similar results were obtained, regardless of the presence or absence of a histidine tag. Also, similar results were obtained even in the case where a combination of antibodies employed was varied; specifically, in the case of an assay system employing 6H11 as an immobilized antibody and 4G6 as a labeled antibody. Therefore, conceivably, such pH-dependent reactivity is attributed not to the properties of an antibody employed, but to an increase in reactivity of OTC with the antibody, which is caused by, for example, change in structure of OTC with pH change, and appearance of an epitope recognized by the antibody.

(4-2) Effect of ProClin Addition on Assay System

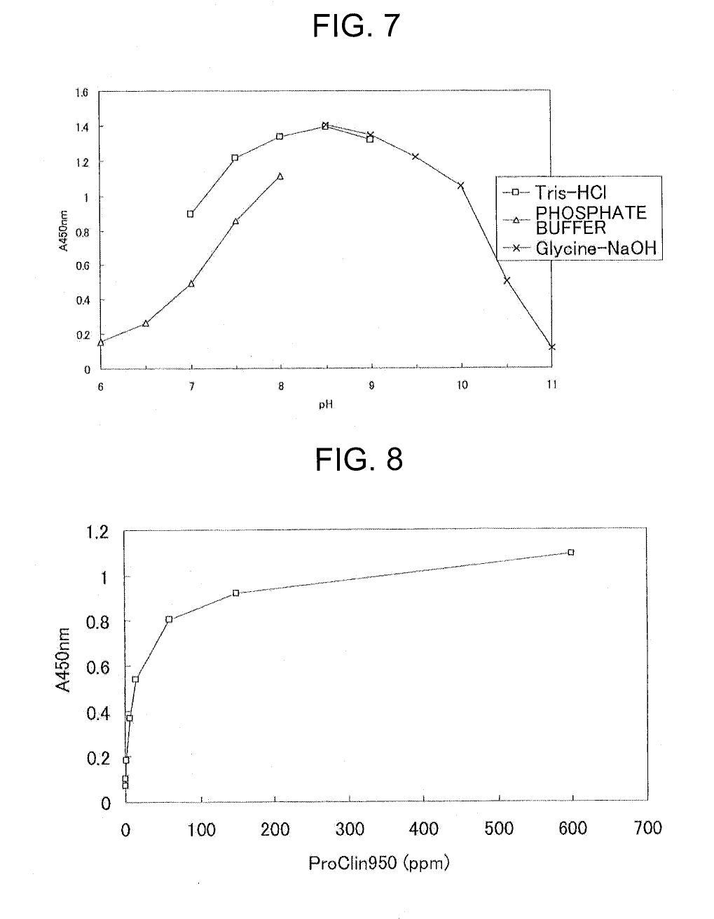

ProClin (available from SUPELCO or Sigma-Aldrich), which is generally employed as an antiseptic in clinical tests, was added to an assay system, and the effect of ProClin on absorbance (reactivity of an antibody) was studied. The results are shown in FIG. 7. As is clear from FIG. 7, unlike the case where ProClin is absent (i.e., sufficient reactivity is obtained only within a pH range of 7.5 to 10.5), when ProClin is added, sufficient reactivity is obtained even within a pH range of 6.5 to 7.5—within which virtually no reactivity has been obtained so far—and reactivity is enhanced within a pH range other than the above pH range. Such results were observed in the case where native OTC was employed, as well as in the case where recombinant OTC was employed. Also, similar results were obtained even in the case where a combination of antibodies employed was varied.

(4-3) Effect of ProClin Concentration

The effect of ProClin concentration on reactivity of an antibody was studied by adding ProClin to an assay system (pH 7.5) at various concentrations. The results are shown in FIG. 8. As is clear from FIG. 8, at pH 7.5, virtually no reactivity is observed in the absence of ProClin, whereas reactivity is increased in accordance with an increase in concentration of added ProClin. Also, at a pH other than 7.5 (e.g., pH 9.5), reactivity was increased in accordance with an increase in ProClin concentration.

(5) Study on Correlation with Enzyme Activity Assay

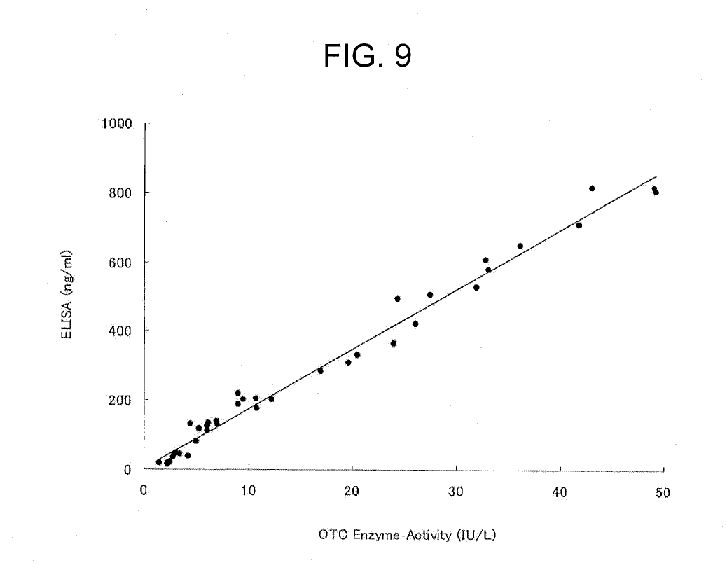

Correlation was studied between serum OTC levels of liver disease patients as measured by means of an enzyme activity assay kit (product of Wako Pure Chemical Industries, Ltd.) and those as measured through the aforementioned ELISA. The results are shown in FIG. 9. As is clear from FIG. 9, the OTC levels as measured through these methods correlate to a very high degree. The standard procedure of the enzyme activity assay kit requires a large amount of serum (i.e., 500 μL), but the aforementioned ELISA requires only a small amount of serum (i.e., 5 μL) for OTC assay. The aforementioned ELISA was found to assay serum OTC levels of liver disease patients at sufficiently high sensitivity through two-hour reaction.

(6) Assay of Healthy Subject Serum

Sera from 94 healthy subjects were assayed. As a result, the average of serum OTC levels of the healthy subjects was found to be about 35 ng/mL, and the maximum serum OTC level of the healthy subjects was found to be about 100 ng/mL. In the cases of healthy subjects positive for another liver disease marker, serum OTC level was found to be 100 ng/mL or more, or higher than the average of serum OTC levels of healthy subjects without hepatic disorders.

It should be understood that various changes and modifications to the presently preferred embodiments described herein will be apparent to those skilled in the art. Such changes and modifications can be made without departing from the spirit and scope of the present application and without diminishing its intended advantages. It is therefore intended that such changes and modifications be covered by the appended claims.

1. A stabilized ornithine transcarbamylase (OTC) solution having a pH of 5.5 to 7.0.

2. An OTC solution as described in claim 1, which further contains at least two members selected from among glycerol, a protein, a substrate for OTC, an OTC reaction product, and an analogue of the substrate or reaction product.

3. An OTC solution as described in claim 2, which is in a liquid state, wherein the protein is a bovine-derived protein.

4. An OTC solution as described in claim 2, which is in a frozen state, wherein the protein is a milk-derived protein.

5. A stabilized, lyophilized OTC product obtained through lyophilization of an OTC solution, wherein a protein and a sugar are incorporated as stabilizers during lyophilization.

6. A lyophilized OTC product as described in claim 5, wherein the OTC solution has a pH of 5.5 to 7.0 before lyophilization.

7. A lyophilized OTC product as described in claim 5, wherein the protein is a bovine-derived protein, and the sugar is a monosaccharide or a disaccharide.

8. An immunological assay method of OTC, the method comprising reacting an OTC antigen with an anti-OTC antibody at a pH of 7.5 to 10.5.

9. A method as described in claim 8, wherein ProClin (trade name) is caused to coexist with an antigen-antibody reaction system.

10. An immunological assay method of OTC, the method comprising reacting an OTC antigen with an anti-OTC antibody at a pH of 6.5 to 10.5 in the presence of ProClin.

11. An OTC detection kit for assaying OTC through a method as recited in any of claims 8 to 10.

12. A kit as described in claim 11, which employs, as a standard substance, an OTC solution or a lyophilized OTC product as recited in any of claims 1 to 7.
